(19) United States
(12) Patent Application Publication
(10) Pub. No.: US 2021/0386914 A1
(43) Pub. Date: Dec. 16, 2021

(54) CORE-SHELL STRUCTURE FOR ESTABLISHING NORMAL AND CANCER ORGANOID MICROENVIRONMENT AND FABRICATION METHOD THEREFOR

(71) Applicant: POSTECH ACADEMY-INDUSTRY FOUNDATION, Pohang-si (KR)

(72) Inventors: Sungjune JUNG, Pohang-si (KR); Hwa Rim LEE, Cheongju-si (KR); Woong Hee YOON, Namyangju-si (KR); Kunyoo SHIN, Pohang-si (KR); Sungeun KIM, Pohang-si (KR); Eunjee KIM, Pohang-si (KR)

(21) Appl. No.: 17/282,809

(22) PCT Filed: Oct. 11, 2019
(86) PCT No.: PCT/KR2019/013332

§ 371 Date: Apr. 5, 2021

(30) Foreign Application Priority Data
Nov. 28, 2018 (KR) 10-2018-0149353

(51) Int. Cl. A61L 27/38; A61L 27/52

(52) U.S. Cl. CPC A61L 27/3813; A61L 27/3895; A61L 2300/64; A61L 2300/62; A61L 27/52

(57) ABSTRACT

Proposed is a core-shell structure including a shell portion and a core portion, in which the shell portion includes n shells that are sequentially located from outside to inside, the core portion includes a core located inside the shell portion, n is any one of natural numbers from 1 to 30, when n is 1, the core is located adjacent to the inside of a first shell, when n is any one of natural numbers from 2 to 30, an n^{th} shell is located adjacent to the inside of an n−1^{th} shell, the n^{th} shell is an empty space or is a hydrogel including at least one of an n^{th} extracellular matrix and an n^{th} cell, the core is an empty space or is a hydrogel including at least one of an extracellular matrix for a core and a cell for a core, two of the n shells and the core that are in contact with each other are not empty spaces simultaneously, and densities of the two of the n shells and the core that are in contact with each other are identical or different, thereby mimicking the construction of hollow organs such as the stomach, intestines, bladder, and lungs.

### TECHNICAL FIELD

The present disclosure relates to a core-shell structure suitable for use in organoids and a method of manufacturing the same, and more particularly to a core-shell structure, which includes a shell portion including n shells that are sequentially located from outside to inside and a core portion including a core that is located inside the shell portion, and in which any one of the n^{th} shell and the core is formed into an empty space, thereby mimicking the overall construction of hollow organs such as the stomach, intestines, and bladder, and a method of manufacturing the same.

### BACKGROUND ART

Bioprinting is a technique for producing a tissue mimic using bioink composed of a hydrogel that mimics a cell and an extracellular matrix.

The types of organisms that play the role of cells in bioink include individual cells, cell masses, cells that are to form a pattern such as normal and cancer organoids, or units containing cells and extracellular matrices.

A normal organoid is a three-dimensional cell structure composed of clusters of cells derived from stem cells, and an organoid exhibits the construction and biological characteristics of part of a biotissue. Intestine and bladder organoids grow in the form of hollow spheres and mimic the epidermal layer thereof. In the organoid construction, the inside of the empty sphere exhibits the characteristics of epithelial cells, and the outside thereof exhibits the characteristics of basal cells. In order to implement a wider range of tissue mimicking, a co-culture method in which components of the corresponding tissue are cultured together with organoids is used.

A cancer (tumor) organoid is derived from cancer cells derived from a patient's cancer tissue, and grows in the form of a solid sphere. It exhibits characteristics such as the gene mutation pattern, anticancer drug reactivity, histopathological characteristics and the like of the derived cancer patient. In cancer tissues, various types of cells, including not only cancer cells but also stromal cells, vascular cells, immune cells and the like, are co-cultured.

Techniques for implementing co-culture include pipetting, printing, hanging drop, and the like. These methods induce interactions between cells, but have limited structural and biological meaning in that they do not realize the three-dimensional construction of tissues.

Therefore, there is a need for a structure capable of mimicking the three-dimensional arrangement of actual tissues and a manufacturing process thereof.

### DISCLOSURE

### Technical Problem

An objective of the present disclosure is to provide a core-shell structure capable of mimicking the three-dimensional arrangement of actual tissues.

Another objective of the present disclosure is to provide a method of manufacturing a core-shell structure capable of mimicking the three-dimensional arrangement of actual tissues.

### Technical Solution

An aspect of the present disclosure provides a core-shell structure including a shell portion and a core portion, in which the shell portion includes n shells that are sequentially located from outside to inside, the core portion includes a core that is located inside the shell portion, n is any one of natural numbers from 1 to 30, when n is 1, the core is located adjacent to the inside of a first shell, when n is any one of natural numbers from 2 to 30, an n^{th} shell is located adjacent to the inside of an n−1^{th} shell, the n^{th} shell is an empty space or is a hydrogel including at least one of an n^{th} extracellular matrix and an n^{th} cell, the core is an empty space or is a hydrogel including at least one of an extracellular matrix for a core and a cell for a core, two of the n shells and the core that are in contact with each other are not empty spaces simultaneously, and the densities of the two of the n shells and the core that are in contact with each other are identical or different.

In addition, n may be 1, the core may be located inside a first shell, the first shell may include a first extracellular matrix and a first cell, the core may include the extracellular matrix for the core and the cell for the core, the first extracellular matrix and the extracellular matrix for the core may be identical to or different from each other, and the first cell and the cell for the core may be identical to or different from each other.

In addition, n may be 2, a second shell may be located inside a first shell, the core may be located inside the second shell, the first shell may include a first extracellular matrix and a first cell, the second shell may include a second extracellular matrix and a second cell, the core may include the extracellular matrix for the core and the cell for the core, the extracellular matrix for the core, the first extracellular matrix, and the second extracellular matrix may be identical to or different from each other, and the cell for the core, the first cell, and the second cell may be identical to or different from each other.

In addition, n may be 2, a second shell may be located inside a first shell, the core may be located inside the second shell, the first shell may include a first extracellular matrix and a first cell, the second shell may be an empty space, the core may include the extracellular matrix for the core and the cell for the core, the extracellular matrix for the core and the first extracellular matrix may be identical to or different from each other, and the cell for the core and the first cell may be identical to or different from each other.

In addition, n may be 2, a second shell may be located inside a first shell, the core may be located inside the second shell, the first shell may include a first extracellular matrix and a first cell, the second shell may include a second extracellular matrix and a second cell, the core may be an empty space, the first extracellular matrix and the second extracellular matrix may be identical to or different from each other, and the first cell and the second cell may be identical to or different from each other.

In addition, the density of the n−1^{th} shell may be lower than the density of the n^{th} shell, and the density of the core may be lower than the density of the first shell.

In addition, at least one of the n shells and the core each independently has at least one shape selected from the group consisting of a spherical shape, a hemispherical shape, a cylinder shape, an elliptical cylinder shape, a cone shape, a truncated cone shape, an elliptical cone shape, a truncated elliptical cone shape, a polygonal prism shape, a polygonal pyramid shape, a truncated polygonal pyramid shape, and combinations thereof.

In addition, each of the extracellular matrix for the core and the n^{th} extracellular matrix may independently include at least one selected from the group consisting of collagen, gelatin, fibrinogen, gelatin methacrylate (GelMA), decellularized extracellular matrix, calcium alginate, Matrigel, nanocellulose, hyaluronic acid, alginate, and elastin.

In addition, each of the cell for the core and the n^{th} cell may independently include at least one selected from the group consisting of a fibroblast, a stem cell, a cancer cell, a vascular cell, a muscle cell, an epidermal cell, an immune cell, a neuron, and a glial cell.

In addition, the fibroblast may include at least one selected from the group consisting of a mammal-derived fibroblast, an alga-derived fibroblast, a reptile-derived fibroblast, an amphibian-derived fibroblast, and a fish-derived fibroblast.

In addition, each of the extracellular matrix for the core and the n^{th} extracellular matrix may independently form a hydrogel through van der Waals attraction, ionic bonding, or covalent bonding.

In addition, the n^{th} cell may include an epidermal cell.

In addition, the epidermal cell may include at least one selected from the group consisting of a keratinocyte and a melanocyte.

In addition, the core-shell structure may be used for an organoid.

Another aspect of the present disclosure provides a method of manufacturing a core-shell structure including a core portion including a core and a shell portion including n shells, including: (a) discharging n−1^{th} bioink to form an n−1^{th} droplet; (b) discharging n^{th} bioink into the n−1^{th} droplet to form an n^{th} droplet inside the n−1^{th} droplet; (c) discharging bioink for a core into the n^{th} droplet to form a core droplet inside the n^{th} droplet; and (d) curing at least one of the core droplet and the n^{th} droplet to form a hydrogel including the core and the shell, in which step (b) is repeated n times, n is any one of natural numbers from 1 to 30, when n is 1, the core droplet is located adjacent to the inside of a first droplet, and when n is any one of natural numbers from 2 to 30, an n^{th} droplet is located adjacent to the inside of an n−1^{th} droplet.

In addition, the method may further include (e) culturing a cell contained in the hydrogel, after step (d).

In addition, the method may further include (f) separating at least one of the core droplet and the n^{th} droplet that is not cured from the hydrogel to form at least one of the core and the n shells into an empty space, after step (d).

In addition, each of the core droplet and the n^{th} droplet that is not cured may independently include at least one selected from the group consisting of collagen, gelatin, Matrigel, calcium alginate, fibrin, and gelatin methacrylate (GelMA).

In addition, the bioink may be discharged through any one process selected from the group consisting of micro-extrusion printing, inkjet printing, laser printing, valve-type printing, spray printing, micro-stamping, and masking.

In addition, the viscosity of the bioink for the core may be 1 to 500 cP, and the viscosity of the n^{th} bioink may be 1 to 500 cP.

### Advantageous Effects

According to the present disclosure, a core-shell structure can be provided in the form of a hollow construction, making it possible to mimic the construction of hollow organs such as the stomach, intestines, bladder, and lungs.

In addition, by increasing the density of the hydrogel constituting the inner layer so as to be higher than that of the outer layer, it is possible to minimize mixing with the outer layer during printing of the hydrogel of the inner layer, and organs in contact with external surfaces, such as the skin, stomach, intestines, bladder, etc., have a characteristic in that the physical strength of the extracellular matrix increases closer to the surface. According to the present disclosure, a structure exhibiting the characteristics of these organs can be manufactured.

In addition, the method of manufacturing the core-shell structure of the present disclosure makes it possible to manufacture the final structure through a single curing process, rather than several curing processes, and during the curing process, hydrogels constituting individual layers are cured together to induce molecular bonding, so the movement and interaction between cells constituting individual layers cannot be inhibited.

In addition, cell patterning to realize a multilayer construction can be implemented using only bioink, without a structural support made of plastic or gelatin on the recessed bottom layer, and by using the process of adding a layer inside a layer, a pattern of three or more layers can be formed in a small structure having a diameter of 5 mm or less.

### BRIEF DESCRIPTION OF DRAWINGS

Since the appended drawings are for reference only to describe exemplary embodiments of the present disclosure, the technical idea of the present disclosure should not be construed as being limited to the accompanying drawings:

FIG. 1 shows a core-shell structure according to the present disclosure;

FIG. 2 schematically shows a process of manufacturing the core-shell structure having various constructions according to an embodiment of the present disclosure;

FIG. 3 shows cross-sectional images of the core-shell structure manufactured in Example 1;

FIG. 4 shows cross-sectional images of the core-shell structure manufactured in Example 2;

FIG. 5 shows a cross-sectional image of the core-shell structure manufactured in Example 3; and

FIG. 6 shows a cross-sectional image of the core-shell structure manufactured in Example 4.

### BEST MODE

Hereinafter, exemplary embodiments of the present disclosure are described in detail with reference to the appended drawings so as to be easily performed by a person having ordinary skill in the art.

However, the following description does not limit the present disclosure to specific embodiments, and moreover, descriptions of known techniques, even if they are pertinent to the present disclosure, are considered unnecessary and may be omitted insofar as they would make the characteristics of the disclosure unclear.

The terms herein are used to explain specific embodiments, and are not intended to limit the present disclosure. Unless otherwise stated, a singular expression includes a plural expression. In this application, the terms “comprise”, “include” or “have” are used to designate the presence of features, numbers, steps, operations, elements, or combinations thereof described in the specification, and should be understood as not excluding the presence or additional possible presence of one or more different features, numbers, steps, operations, elements, or combinations thereof.

As used herein, the terms “first”, “second”, etc. may be used to describe various elements, but these elements are not to be limited by these terms. These terms are only used to distinguish one element from another. For example, a first element may be termed a second element, and similarly, a second element may be termed a first element, without departing from the scope of the present disclosure.

Further, it will be understood that when an element is referred to as being “formed” or “stacked” on another element, it can be formed or stacked so as to be directly attached to all surfaces or to one surface of the other element, or intervening elements may be present therebetween.

Hereinafter, a detailed description will be given of a core-shell structure and a method of manufacturing the same according to the present disclosure, which is set forth to illustrate but is not to be construed as limiting the present disclosure, and the present disclosure is defined only by the accompanying claims.

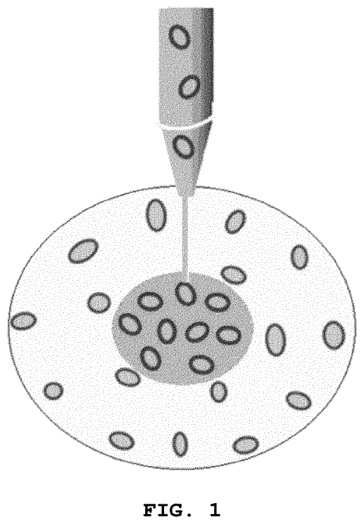

FIG. 1 shows a core-shell structure according to the present disclosure.

With reference to FIG. 1, the core-shell structure includes a shell portion and a core portion, in which the shell portion includes n shells that are sequentially located from outside to inside, the core portion includes a core that is located inside the shell portion, n is any one of natural numbers from 1 to 30, when n is 1, the core is located adjacent to the inside of a first shell, when n is any one of natural numbers from 2 to 30, an n^{th} shell is located adjacent to the inside of an n−1^{th} shell, the n^{th} shell is an empty space or is a hydrogel including at least one of an n^{th} extracellular matrix and an n^{th} cell, the core is an empty space or is a hydrogel including at least one of an extracellular matrix for a core and a cell for a core, two of the n shells and the core that are in contact with each other are not empty spaces simultaneously, and the densities of the two of the n shells and the core that are in contact with each other are identical or different.

In addition, n may be 1, the core may be located inside a first shell, the first shell may include a first extracellular matrix and a first cell, the core may include the extracellular matrix for the core and the cell for the core, the first extracellular matrix and the extracellular matrix for the core may be identical to or different from each other, and the first cell and the cell for the core may be identical to or different from each other.

In addition, n may be 2, a second shell may be located inside a first shell, the core may be located inside the second shell, the first shell may include a first extracellular matrix and a first cell, the second shell may include a second extracellular matrix and a second cell, the core may include the extracellular matrix for the core and the cell for the core, the extracellular matrix for the core, the first extracellular matrix, and the second extracellular matrix may be identical to or different from each other, and the cell for the core, the first cell, and the second cell may be identical to or different from each other.

In addition, n may be 2, a second shell may be located inside a first shell, the core may be located inside the second shell, the first shell may include a first extracellular matrix and a first cell, the second shell may be an empty space, the core may include the extracellular matrix for the core and the cell for the core, the extracellular matrix for the core and the first extracellular matrix may be identical to or different from each other, and the cell for the core and the first cell may be identical to or different from each other.

In addition, n may be 2, a second shell may be located inside a first shell, the core may be located inside the second shell, the first shell may include a first extracellular matrix and a first cell, the second shell may include a second extracellular matrix and a second cell, the core may be an empty space, the first extracellular matrix and the second extracellular matrix may be identical to or different from each other, and the first cell and the second cell may be identical to or different from each other.

In addition, the density of the n−1^{th} shell may be lower than the density of the n^{th} shell, and the density of the core may be lower than the density of the first shell.

In addition, at least one of the n shells and the core each independently has at least one shape selected from the group consisting of a spherical shape, a hemispherical shape, a cylinder shape, an elliptical cylinder shape, a cone shape, a truncated cone shape, an elliptical cone shape, a truncated elliptical cone shape, a polygonal prism shape, a polygonal pyramid shape, a truncated polygonal pyramid shape, and combinations thereof, and preferably has a spherical shape.

In addition, each of the extracellular matrix for the core and the n^{th} extracellular matrix may independently include at least one selected from the group consisting of collagen, gelatin, fibrinogen, gelatin methacrylate (GelMA), decellularized extracellular matrix, calcium alginate, Matrigel, nanocellulose, hyaluronic acid, alginate, and elastin, and preferably includes at least one selected from among collagen, gelatin, fibrinogen, gelatin methacrylate, decellularized extracellular matrix, calcium alginate, and Matrigel, and more preferably collagen and gelatin.

In addition, each of the cell for the core and the n^{th} cell may independently include at least one selected from the group consisting of a fibroblast, a stem cell, a cancer cell, a vascular cell, a muscle cell, an epidermal cell, an immune cell, a neuron, and a glial cell, and preferably includes a fibroblast.

In addition, the fibroblast may include at least one selected from the group consisting of a mammal-derived fibroblast, an alga-derived fibroblast, a reptile-derived fibroblast, an amphibian-derived fibroblast, and a fish-derived fibroblast, and preferably includes a mammal-derived fibroblast.

In addition, each of the extracellular matrix for the core and the n^{th} extracellular matrix may independently form a hydrogel through van der Waals attraction, ionic bonding, or covalent bonding.

In addition, the n^{th} cell may include an epidermal cell.

In addition, the epidermal cell may include at least one selected from the group consisting of a keratinocyte and a melanocyte.

In addition, the core-shell structure may be used for an organoid.

A method of manufacturing the core-shell structure according to the present disclosure is described below.

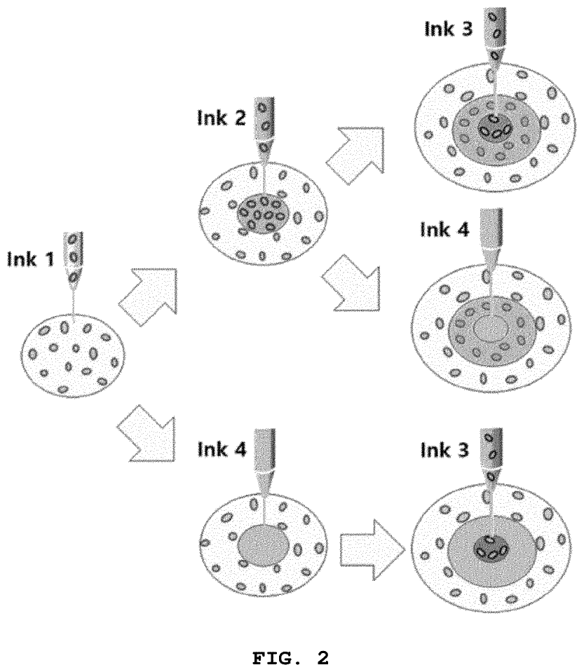

FIG. 2 shows the process of manufacturing the core-shell structure having various constructions according to the present disclosure.

With reference to FIG. 2, the method of manufacturing the core-shell structure including a core portion including a core and a shell portion including n shells includes (a) discharging n−1^{th} bioink to form an n−1^{th} droplet, (b) discharging n^{th} bioink into the n−1^{th} droplet to form an n^{th} droplet inside the n−1^{th} droplet, (c) discharging bioink for a core into the n^{th} droplet to form a core droplet inside the n^{th} droplet, and (d) curing at least one of the core droplet and the n^{th} droplet to form a hydrogel including the core and the shell, in which step (b) is repeated n times, n is any one of natural numbers from 1 to 30, when n is 1, the core droplet is located adjacent to the inside of a first droplet, and when n is any one of natural numbers from 2 to 30, an n^{th} droplet is located adjacent to the inside of an n−1^{th} droplet.

In addition, the method may further include (e) culturing a cell contained in the hydrogel, after step (d).

In addition, the method may further include (f) separating at least one of the core droplet and the n^{th} droplet that is not cured from the hydrogel to form at least one of the core and the n shells into an empty space, after step (d).

In addition, each of the core droplet and the n^{th} droplet that is not cured may independently include at least one selected from the group consisting of collagen, gelatin, Matrigel, calcium alginate, fibrin, and gelatin methacrylate (GelMA), and preferably includes gelatin.

In addition, the bioink may be discharged through any one process selected from the group consisting of micro-extrusion printing, inkjet printing, laser printing, valve-type printing, spray printing, micro-stamping, and masking, and is preferably discharged through any one process selected from among micro-extrusion printing and inkjet printing, and more preferably through micro-extrusion printing.

In addition, the viscosity of the bioink for the core may be 1 to 500 cP, and the viscosity of the n^{th} bioink may be 1 to 500 cP.

If the viscosity of the bioink for the core is less than 1 cP, the construction after printing cannot be maintained, which is undesirable, whereas if the viscosity thereof exceeds 500 cP, the nozzle of the printer may be clogged when printing the bioink, which is undesirable.

If the viscosity of the n^{th} bioink is less than 1 cP, the construction after printing cannot be maintained, which is undesirable, whereas if the viscosity thereof exceeds 500 cP, the nozzle of the printer may be clogged when printing the bioink, which is undesirable.

With reference to FIG. 2, a core-shell structure having one shell may be manufactured by discharging bioink 1 (Ink 1) containing a cell and then inserting only bioink 2 (Ink 2) containing a cell. In addition, a core-shell structure having two shells may be manufactured by discharging bioink 2 (Ink 2) into bioink 1 (Ink 1) and discharging bioink 3 (Ink 3) containing a cell into the bioink 2 (Ink 2).

With reference to FIG. 2, a structure is manufactured by discharging bioink 2 (Ink 2) into bioink 1 (Ink 1), inserting bioink 4 (Ink 4) composed of gelatin into the bioink 2 (Ink 2), and performing a curing process. Thereafter, when the structure is placed under culture conditions for 24 hours during the subsequent culture process, the gelatin component escapes therefrom to form an empty space, so a core-shell structure having two shells and an empty core may be manufactured.

With reference to FIG. 2, a structure is manufactured by inserting bioink 4 (Ink 4) composed of gelatin into bioink 1 (Ink 1), discharging bioink 2 (Ink 2) into the bioink 4 (Ink 4), and performing a curing process. Thereafter, when the structure is placed under culture conditions for 24 hours during the subsequent culture process, the gelatin component escapes therefrom to form an empty space, thus manufacturing a core-shell structure having two shells in which the second shell is empty.

### MODE FOR DISCLOSURE

### Examples

A better understanding of the present disclosure may be obtained through the following examples. However, these examples are merely set forth to illustrate the present disclosure, and are not to be construed as limiting the scope of the present disclosure.

### Manufacture of Bioink

### Preparation Example 1

Bioink having a density of 5×10⁶ cells/ml and a viscosity of 20 cp was manufactured by performing a mixing process such that 1×10⁷ cells of mouse-derived fibroblast (NIH/3T3) stained with blue were uniformly distributed in 1.0 w/v % neutral (pH 7) collagen.

### Preparation Example 2

Bioink was manufactured in the same manner as in Preparation Example 1, with the exception that the fibroblast was stained with green, rather than being stained with blue.

### Preparation Example 3

Bioink was manufactured in the same manner as in Preparation Example 1, with the exception that the fibroblast was stained with red, rather than being stained with blue.

### Example 1: Core-Shell Structure Having One Shell

With reference to FIGS. 1 and 2, 30 μL of first bioink was discharged to the center of a piece of Parafilm, which was recessed in a hemispherical shape having a diameter of about 5 mm, thus forming a first droplet in the shape of a flat sphere. Here, as the first bioink, the bioink manufactured according to Preparation Example 2 was used.

The bioink manufactured according to Preparation Example 3 was used as bioink for a core. A core droplet was formed inside the first droplet by discharging 10 μL of the bioink for the core to the center portion of the first droplet.

After completion of discharge, the core droplet and the first droplet were placed in an incubator at 37° C. and cured for 1 hour, thus forming a hydrogel including the core and the shell.

The cured structure was placed in Dulbecco's Modified Eagle's Medium (DMEM) containing 10 v/v % fetal bovine serum (FBS), 1 v/v % penicillin and streptomycin antibiotics and cultured, thereby manufacturing a core-shell structure having one shell.

### Example 2: Core-Shell Structure Having Two Shells

With reference to FIGS. 1 and 2, 30 μL of first bioink was discharged to the center of a piece of Parafilm, which was recessed in a hemispherical shape having a diameter of about 5 mm, thus forming a first droplet in the shape of a flat sphere. Here, as the first bioink, the bioink manufactured according to Preparation Example 1 was used.

The bioink manufactured according to Preparation Example 2 was used as second bioink. A second droplet was formed inside the first droplet by discharging 10 μL of the second bioink to the center portion of the first droplet.

The bioink manufactured according to Preparation Example 3 was used as bioink for a core. A core droplet was formed inside the second droplet by discharging 2 μL of the bioink for the core to the center portion of the second droplet.

After completion of discharge, the core droplet, the first droplet and the second droplet were placed in an incubator at 37° C. and cured for 1 hour, thus forming a hydrogel including the core and the shell.

The cured structure was placed in Dulbecco's Modified Eagle's Medium (DMEM) containing 10 v/v % fetal bovine serum (FBS), 1 v/v % penicillin and streptomycin antibiotics and cultured, thereby manufacturing a core-shell structure having two shells.

### Example 3: Core-Shell Structure Having Two Shells and Empty Second Shell

With reference to FIGS. 1 and 2, 30 μL of first bioink was discharged to the center of a piece of Parafilm, which was recessed in a hemispherical shape having a diameter of about 5 mm, thus forming a first droplet in the shape of a flat sphere. Here, as the first bioink, the bioink manufactured according to Preparation Example 1 was used.

A 20 w/v % neutral (pH 7) gelatin solution at 4° C. was used as second bioink. 10 μL of the second bioink was discharged, thus forming a second droplet inside the first droplet.

The bioink manufactured according to Preparation Example 3 was used as bioink for a core. A core droplet was formed inside the second droplet by discharging 2 μL of the bioink for the core to the center portion of the second droplet.

After completion of discharge, the core droplet, the first droplet and the second droplet were placed in an incubator at 37° C., and the core droplet and the first droplet composed of collagen were cured for 1 hour. The second droplet composed of gelatin was not cured.

The cured structure was placed in Dulbecco's Modified Eagle's Medium (DMEM) containing 10 v/v % fetal bovine serum (FBS), 1 v/v % penicillin and streptomycin antibiotics and cultured. Culture was carried out for 24 hours to allow the uncured second droplet to escape therefrom, thereby manufacturing a core-shell structure having two shells and the empty second shell.

### Example 4: Core-Shell Structure Having Two Shells and Empty Core

With reference to FIGS. 1 and 2, 30 μL of first bioink was discharged to the center of a piece of Parafilm, which was recessed in a hemispherical shape having a diameter of about 5 mm, thus forming a first droplet in the shape of a flat sphere. Here, as the first bioink, the bioink manufactured according to Preparation Example 3 was used.

The bioink manufactured according to Preparation Example 2 was used as second bioink. 10 μL of the second bioink was discharged to the center portion of the first droplet, thus forming a second droplet inside the first droplet.

A 20 w/v % neutral (pH 7) gelatin solution at 4° C. was used as bioink for a core. A core droplet was formed inside the second droplet by discharging 2 μL of the bioink for the core to the center portion of the second droplet.

After completion of discharge, the core droplet, the first droplet and the second droplet were placed in an incubator at 37° C., and the first droplet and the second droplet composed of collagen were cured for 1 hour. The core droplet composed of gelatin was not cured.

The cured structure was placed in Dulbecco's Modified Eagle's Medium (DMEM) containing 10 v/v % fetal bovine serum (FBS), 1 v/v % penicillin and streptomycin antibiotics and cultured. Culture was carried out for 24 hours to allow the uncured core droplet to escape therefrom, thereby manufacturing a core-shell structure having two shells and the empty core.

### Test Example

### Test Example 1: Cross-Sectional Image of Core-Shell Structure

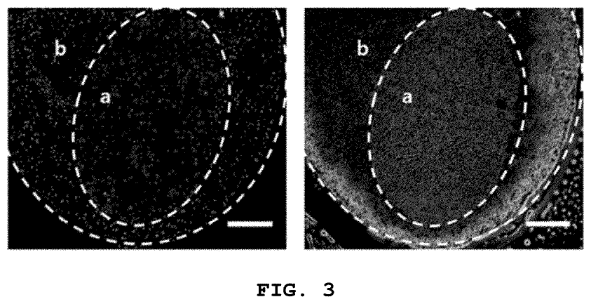

FIGS. 3 to 6 are cross-sectional images of the core-shell structures manufactured according to Examples 1 to 4, obtained using a fluorescence microscope.

Cross-Sectional Images of Core-Shell Structure Having One Shell

FIG. 3 shows the cross-sectional images of the core-shell structure manufactured according to Example 1. With reference to FIG. 3, in the left fluorescence image, it can be seen that the core portion a and the shell portion b are clearly distinguishable, and in the right image, it can be seen that the distinguishable layer represents the boundary between the discharged inner layer and the outer layer.

Cross-Sectional Images of Core-Shell Structure Having Two Shells

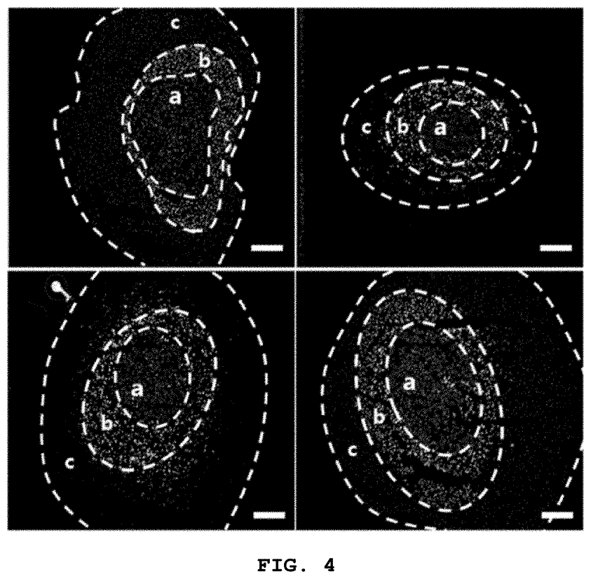

FIG. 4 shows the cross-sectional images of the core-shell structure manufactured according to Example 2. With reference to FIG. 4, it can be seen that the core-shell structure manufactured according to Example 2 had the red core a, the green second shell b, and the blue first shell c, based on which the formation of the core-shell structure having two shells according to Example 2 was confirmed.

Cross-Sectional Image of Core-Shell Structure Having Two Shells and Empty Second Shell

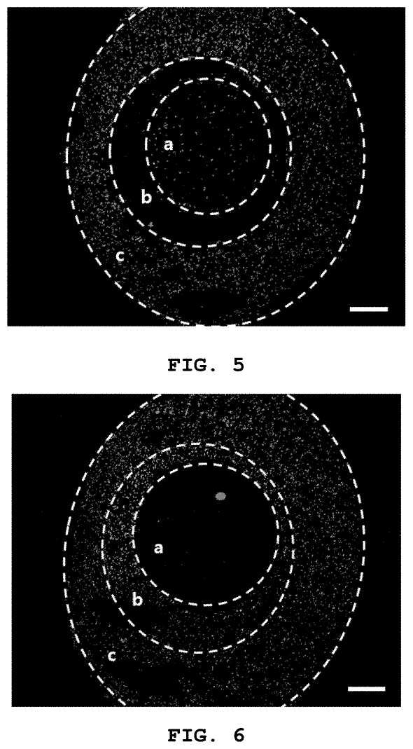

FIG. 5 shows the cross-sectional image of the core-shell structure manufactured according to Example 3. With reference to FIG. 5, it can be seen that a structure including blue c, black b, and red a from the outside to the inside was formed. The red portion a is the core, and the blue portion c and the black portion b represent the first shell and the second shell, respectively. The red core a and the blue first shell c showed cells arranged therein, and the black second shell b was observed to be empty. Therefore, it can be confirmed that the core-shell structure having two shells and the empty second shell according to Example 3 was formed.

Cross-Sectional Image of Core-Shell Structure Having Two Shells and Empty Core

FIG. 6 shows the cross-sectional image of the core-shell structure manufactured according to Example 4. With reference to FIG. 6, it can be seen that a structure including red c, green b, and black a from the outside to the inside was formed. The black portion a is the core, and the red portion c and the green portion b represent the first shell and the second shell, respectively. The red first shell c and the green second shell b showed cells arranged therein, and the black core a was observed to be empty. Therefore, it can be confirmed that the core-shell structure having two shells and the empty core according to Example 4 was formed.

The scope of the present disclosure is represented by the claims below rather than the aforementioned detailed description, and all changes or modified forms that are capable of being derived from the meaning, range, and equivalent concepts of the appended claims should be construed as being included in the scope of the present disclosure.

### INDUSTRIAL APPLICABILITY

According to the present disclosure, the core-shell structure can be provided in the form of a hollow construction, making it possible to mimic the construction of hollow organs such as the stomach, intestines, bladder, and lungs.

In addition, by increasing the density of the hydrogel constituting the inner layer so as to be higher than that of the outer layer, it is possible to minimize mixing with the outer layer during printing of the hydrogel of the inner layer. Organs in contact with external surfaces, such as the skin, stomach, intestines, bladder, etc. have a characteristic in that the physical strength of the extracellular matrix increases closer to the surface. According to the present disclosure, a structure exhibiting the characteristics of these organs can be manufactured.

In addition, the method of manufacturing the core-shell structure of the present disclosure enables the final structure to be formed through a single curing process, rather than several curing processes. During the curing process, hydrogels constituting individual layers are cured together to induce molecular bonding, so the movement and interaction between cells constituting individual layers cannot be inhibited.

In addition, cell patterning to realize a multilayer construction can be implemented using only bioink, without a structural support made of plastic or gelatin on the recessed bottom layer. By using the process of adding a layer inside a layer, a pattern of three or more layers can be formed in a small structure having a diameter of 5 mm or less.

1. A core-shell structure comprising a shell portion and a core portion,
wherein the shell portion comprises n shells that are sequentially located from outside to inside,
the core portion comprises a core that is located inside the shell portion,
n is any one of natural numbers from 1 to 30,
when n is 1, the core is located adjacent to an inside of a first shell,
when n is any one of natural numbers from 2 to 30, an n^{th} shell is located adjacent to an inside of an n−1^{th} shell,
the n^{th} shell is an empty space, or is a hydrogel comprising at least one of an n^{th} extracellular matrix and an n^{th} cell,
the core is an empty space, or is a hydrogel comprising at least one of an extracellular matrix for a core and a cell for a core,
two of the n shells and the core that are in contact with each other are not empty spaces simultaneously, and
densities of the two of the n shells and the core that are in contact with each other are identical or different.

2. The core-shell structure of claim 1, wherein
n is 1,
the core is located inside a first shell,
the first shell comprises a first extracellular matrix and a first cell,
the core comprises the extracellular matrix for the core and the cell for the core,
the first extracellular matrix and the extracellular matrix for the core are identical to or different from each other, and
the first cell and the cell for the core are identical to or different from each other.

3. The core-shell structure of claim 1, wherein
n is 2,
a second shell is located inside a first shell,
the core is located inside the second shell,
the first shell comprises a first extracellular matrix and a first cell,
the second shell comprises a second extracellular matrix and a second cell,
the core comprises the extracellular matrix for the core and the cell for the core,
the extracellular matrix for the core, the first extracellular matrix, and the second extracellular matrix are identical to or different from each other, and
the cell for the core, the first cell, and the second cell are identical to or different from each other.

4. The core-shell structure of claim 1, wherein
n is 2,
a second shell is located inside a first shell,
the core is located inside the second shell,
the first shell comprises a first extracellular matrix and a first cell,
the second shell is an empty space,
the core comprises the extracellular matrix for the core and the cell for the core,
the extracellular matrix for the core and the first extracellular matrix are identical to or different from each other, and
the cell for the core and the first cell are identical to or different from each other.

5. The core-shell structure of claim 1, wherein
n is 2,
a second shell is located inside a first shell,
the core is located inside the second shell,
the first shell comprises a first extracellular matrix and a first cell,
the second shell comprises a second extracellular matrix and a second cell,
the core is an empty space,
the first extracellular matrix and the second extracellular matrix are identical to or different from each other, and
the first cell and the second cell are identical to or different from each other.

6. The core-shell structure of claim 1, wherein a density of the n−1^{th} shell is lower than a density of the n^{th} shell, and a density of the core is lower than a density of the first shell.

7. The core-shell structure of claim 1, wherein at least one of the n shells and the core each independently has at least one shape selected from the group consisting of a spherical shape, a hemispherical shape, a cylinder shape, an elliptical cylinder shape, a cone shape, a truncated cone shape, an elliptical cone shape, a truncated elliptical cone shape, a polygonal prism shape, a polygonal pyramid shape, a truncated polygonal pyramid shape, and combinations thereof.

8. The core-shell structure of claim 1, wherein each of the extracellular matrix for the core and the n^{th} extracellular matrix independently comprises at least one selected from the group consisting of collagen, gelatin, fibrinogen, gelatin methacrylate (GelMA), decellularized extracellular matrix, calcium alginate, Matrigel, nanocellulose, hyaluronic acid, alginate, and elastin.

9. The core-shell structure of claim 1, wherein each of the cell for the core and the n^{th} cell independently comprises at least one selected from the group consisting of a fibroblast, a stem cell, a cancer cell, a vascular cell, a muscle cell, an epidermal cell, an immune cell, a neuron, and a glial cell.

10. The core-shell structure of claim 9, wherein the fibroblast comprises at least one selected from the group consisting of a mammal-derived fibroblast, an alga-derived fibroblast, a reptile-derived fibroblast, an amphibian-derived fibroblast, and a fish-derived fibroblast.

11. The core-shell structure of claim 1, wherein each of the extracellular matrix for the core and the n^{th} extracellular matrix independently forms a hydrogel through van der Waals attraction, ionic bonding, or covalent bonding.

12. The core-shell structure of claim 1, wherein the n^{th} cell comprises an epidermal cell.

13. The core-shell structure of claim 12, wherein the epidermal cell comprises at least one selected from the group consisting of a keratinocyte and a melanocyte.

14. The core-shell structure of claim 1, wherein the core-shell structure is used for an organoid.

15. A method of manufacturing a core-shell structure comprising a core portion comprising a core and a shell portion comprising n shells, comprising:
(a) discharging n−1^{th} bioink to form an n−1^{th} droplet;
(b) discharging n^{th} bioink into the n−1^{th} droplet to form an n^{th} droplet inside the n−1^{th} droplet;
(c) discharging bioink for a core into the n^{th} droplet to form a core droplet inside the n^{th} droplet; and
(d) curing at least one of the core droplet and the n^{th} droplet to form a hydrogel comprising the core and the shell,
wherein step (b) is repeated n times,
n is any one of natural numbers from 1 to 30,
when n is 1, the core droplet is located adjacent to an inside of a first droplet, and
when n is any one of natural numbers from 2 to 30, an n^{th} droplet is located adjacent to an inside of an n−1^{th} droplet.

16. The method of claim 15, further comprising (e) culturing a cell contained in the hydrogel, after step (d).

17. The method of claim 15, further comprising (f) separating at least one of the core droplet and the n^{th} droplet that is not cured from the hydrogel to form at least one of the core and the n shells into an empty space, after step (d).

18. The method of claim 17, wherein each of the core droplet and the n^{th} droplet that is not cured independently comprises at least one selected from the group consisting of gelatin, Matrigel, calcium alginate, fibrin, and gelatin methacrylate (GelMA).

19. The method of claim 15, wherein the bioink is discharged through any one process selected from the group consisting of micro-extrusion printing, inkjet printing, laser printing, valve-type printing, spray printing, micro-stamping, and masking.

20. The method of claim 15, wherein a viscosity of the bioink for the core is 1 to 500 cP, and a viscosity of the n^{th} bioink is 1 to 500 cP.
